(19) United States
(12) Patent Application Publication
(10) Pub. No.: US 2016/0081613 A1
(43) Pub. Date: Mar. 24, 2016

(54) ARRANGEMENT AND METHOD FOR OUTPUTTING LIGHT SIGNALS AT A MEDICAL-TECHNICAL INSTALLATION

(71) Applicants: Christoph Braun, Rosenheim (DE); Bastian Rackow, Erlangen (DE)

(72) Inventors: Christoph Braun, Rosenheim (DE); Bastian Rackow, Erlangen (DE)

(21) Appl. No.: 14/857,352

(22) Filed: Sep. 17, 2015

(30) Foreign Application Priority Data
Sep. 18, 2014 (DE) 102014218826.1

(51) Int. Cl. A61B 5/00; A61B 6/03; A61B 6/00; A61B 19/00; A61B 5/08; A61M 21/02; A61B 5/021; A61B 5/145; A61B 5/01; A61B 5/053; A61B 5/0488; A61B 5/0476; H05B 37/02; A61B 5/024

(52) U.S. Cl. CPC A61B 5/486; H05B 37/0227; A61B 6/035; A61B 6/44; A61B 19/52; A61B 5/7271; A61B 5/7264; A61B 5/08; A61B 5/024; A61B 5/021; A61B 5/14542; A61B 5/01; A61B 5/0531; A61B 5/0488; A61B 5/0476; A61M 21/02

(57) ABSTRACT

The principle of biofeedback is used to incorporate a patient into a control loop for relaxation by light signals. For this purpose, a sensor measures physiological parameters of the patient. By virtue of the fact that the respective physiological parameter controls a lighting parameter such as light color or light intensity of a lighting unit using the signal processing of a microprocessor in a control loop, the patient acquires visual biofeedback with respect to his/her physiological parameter. Endogenous regulation processes of the patient are thus made accessible to his/her consciousness. The outputting of the light signals is continued until the patient is prepared for the required examination and there is no need for a further administration of medicaments for calming. The work results of the medical-technical installation may be kept optimal as a result of the relaxation of the patient even without administration of medicaments.

This application claims the benefit of DE 10 2014 218 826.1, filed on Sep. 18, 2014, which is hereby incorporated by reference in its entirety.

### BACKGROUND

The present embodiments relate to an arrangement and a method for outputting light signals at a medical-technical installation.

A computed tomography apparatus substantially includes a round gantry, in which a patient to be examined is positioned by the patient being moved on a patient table into the gantry. The examination area, the computed tomography apparatus, and the entire surroundings are unfamiliar to many patients. Many of the patients feel uncertain with regard to their health. These circumstances have the effect that the patient experiences feelings of uncertainty, anxiety and fear, which are also brought about by the confined situation in the gantry. The radiological examination is therefore felt to be unpleasant or even threatening. However, the anxiety and fear may adversely influence the quality of the examination. By way of example, the patient's nervousness may have the effect that, in thoracic examinations, the patient may not hold his/her breath for a sufficiently long period of time. Moreover, the patient's pulse may be too high for cardiological examinations owing to his/her agitation.

It is known for trained personnel to calm the patient beforehand with a great deal of time being expended and in a leisurely way. In some instances, medicaments for calming the patient are also administered.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an arrangement and a method that contribute to the calming of the patient in a technical way are provided.

An arrangement for outputting light signals at a medical-technical installation includes at least one lighting unit having at least one lighting parameter that is controllable. The arrangement also includes at least one sensor configured for measuring at least one physiological parameter of a patient and for generating a signal depending on the physiological parameter. The arrangement includes at least one microprocessor programmed for evaluating the signal and for controlling the lighting parameter of the lighting unit depending on the signal. The arrangement also includes a medical-technical installation configured for an examination or treatment of the patient in a different way than with the lighting unit and the sensor.

In the method for outputting light signals at a medical-technical installation, at least one sensor measures at least one physiological parameter of a patient and generates a signal depending on the physiological parameter. At least one microprocessor evaluates the signal and controls at least one lighting parameter of at least one lighting unit depending on the signal. The lighting unit is arranged at or alongside a medical-technical installation, which is configured for an examination or treatment of the patient in a different way than with the lighting unit and the sensor.

The advantages mentioned below may also be advantages that are afforded only by individual embodiments, variants, or developments.

The arrangement and the method give rise to a control loop that also includes the patient. The principle of biofeedback is used in this case. Biofeedback is a non-medicinal method for influencing the autonomic nervous system. For this purpose, the sensor measures physiological parameters of the patient. Since the respective physiological parameter controls the lighting parameter of the lighting unit by the signal processing of the microprocessor in a control loop, the patient acquires visual biofeedback with respect to his/her physiological parameter. Endogenous regulation processes of the patient are thus made accessible to his/her consciousness. As a result of this, the patient may relax. In this case, the patient learns to control his/her own vegetative state via the visual feedback. As a result, the patient is progressively calmed to a greater and greater extent. Trained personnel may amplify the perception and the conscious processing of the biofeedback by pointing out the light to the patient or instructing the patient to breathe with the rhythm of the light signal, for example. The outputting of the light signals is continued until the patient is prepared for the required examination and there is no need for a further administration of medicaments for calming.

As a result of the biofeedback via the lighting unit that is provided by the arrangement and the method, patients' behavior during the examination is more cooperative. The time expenditure for the examination and the care effort for the personnel are reduced. The patient's individual sensitivities are nevertheless catered for by the arrangement and the method. The work results of the medical-technical installation may be kept optimal as a result of the relaxation of the patient even without administration of medicaments. The use of medicaments may be at least reduced by virtue of the better compliance of the patient. As a result of this, side effects of the administration of medicaments are also reduced or entirely precluded. The patient's wellbeing during the examination is also increased.

In one development, the lighting unit is mounted flat on a surface of the medical-technical installation. Alternatively, the lighting unit illuminates the surface of the medical-technical installation.

This development takes account of the fact that a patient's field of view in a gantry is almost completely filled by the medical-technical installation.

In accordance with one embodiment, the microprocessor continuously controls the lighting parameter depending on a fluctuating quantitative variable determined by the microprocessor by continuous evaluation of the signal.

In one development, the microprocessor evaluates the signal in time intervals, classifies the signal into one from a plurality of categories, and controls the lighting parameter depending on the classified category.

The categories are, for example, different physiological states that may be unambiguously derived from one or more physiological parameters, such as, for example, “agitated”, “sleepy” or “relaxed”. Each of these categories may be assigned a dedicated light program that the microprocessor loads from an electronic memory and outputs via the lighting unit.

In accordance with one embodiment, the sensor measures a rhythmic physiological process. The microprocessor controls the lighting parameter depending on a frequency or amplitude of the signal. Alternatively, the microprocessor controls the lighting parameter depending on a change in the frequency or amplitude of the signal. In accordance with a further alternative, the microprocessor controls the lighting parameter depending on a variance of the frequency or amplitude of the signal.

Frequency and variance of heartbeat or respiration are meaningful physiological parameters that may be evaluated for the biofeedback.

In one development, the lighting parameter is a color temperature, a spectral composition, or a light intensity.

A control of the color temperature or of the spectral composition (e.g., of the light color) may be realized particularly well by LED luminaires.

In accordance with one embodiment, the sensor measures a respiration, a heartbeat, a blood pressure, a blood oxygen content, a skin temperature, a skin resistance, a muscle potential, or a brainwave.

Suitable sensors include, for example, sensors for ECG, EMG, EOG or EEG.

In one development, the medical-technical installation is a radiological imaging installation (e.g., a computed tomography installation or a C-arm X-ray apparatus) or an installation for irradiating a patient (e.g., a medical linear accelerator).

A computer program including instructions is stored on a non-transitory computer-readable storage medium. The method is executed if the method is processed in the microprocessor.

The computer program is processed in the microprocessor and executes the method in the process.

### BRIEF DESCRIPTION OF THE DRAWING

FIG. 1 shows an exemplary embodiment of an arrangement for outputting light signals at a medical-technical installation.

### DETAILED DESCRIPTION

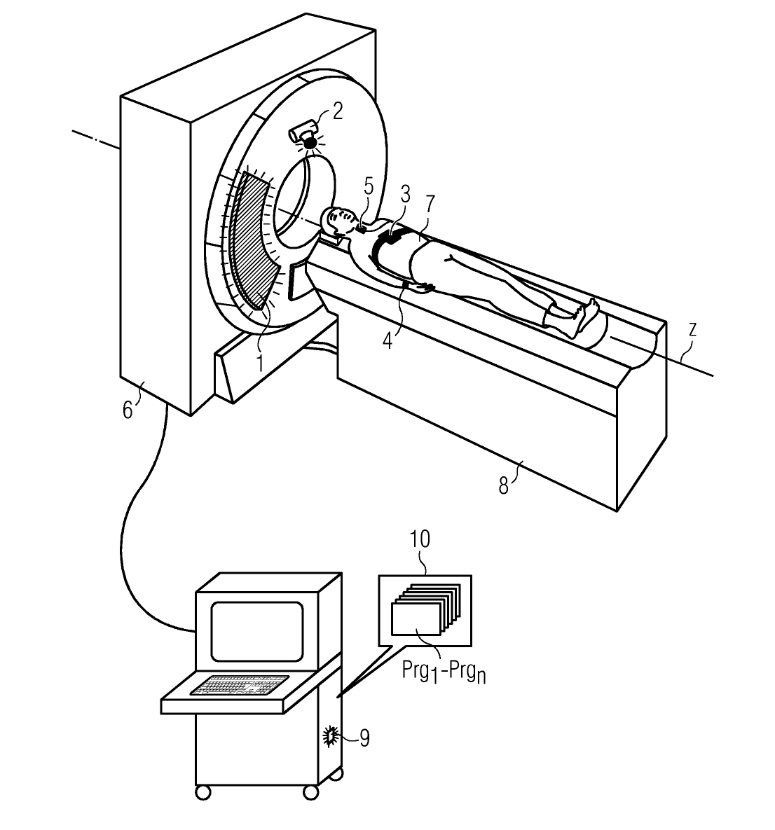

FIG. 1 shows one embodiment of a medical-technical installation 6 (e.g., a computed tomography apparatus including a tubular gantry). A patient 7 lies on a patient table 8 and is introduced into the gantry in the z-direction for examination. Two lighting units 1, 2 are mounted on the medical-technical installation 6 such that the light emitted by the two lighting units 1, 2 appears in the field of view of the patient 7. For this purpose, any desired lighting units 1, 2 may be used. The lighting units 1, 2, may be configured, if appropriate, with regard to electrotechnical boundary conditions on the medical-technical installation 6. In principle, the lighting units 1, 2 used may be, for example, LED lamps or planar LED panels since these enable the light color to be controlled. The respective lighting unit 1, 2 may also be equipped with a projection lens and irradiate the medical-technical installation 6 from a remote position such that light is reflected by the medical-technical installation 6 within the field of view of the patient 7.

The patient 7 wears sensors 3, 4, 5 (e.g., a chest strap for measuring his/her respiration or an ECG, an electrode on the neck for EMG measurement, electrodes on the head for measuring an EOG or an EEG, or a sensor for measuring a skin temperature, a skin resistance, a blood pressure or a blood oxygen content). The medical-technical installation 6 is connected to a display and operating unit incorporating a microprocessor 9 that accesses an electronic memory 10 in which light programs PRG₁ to PRGₙ are stored.

The lighting units 1, 2 provide a light installation at the medical-technical installation 6 having a real light that is readily visible to the patient 7 since coloration is imparted to large parts of the medical-technical installation 6. The microprocessor 9 couples at least one lighting parameter of the lighting units 1, 2, (e.g., a color temperature, a spectral composition or light color, or a light intensity) to a vegetative state of the patient 7 that is measured by the sensors 3, 4, 5 by corresponding physiological parameters. Depending on the state of the patient 7, the microprocessor 9 chooses one of the light programs PRG₁ to PRGₙ, stored in the electronic memory 10, for outputting light signals via the lighting units 1, 2. Alternatively or additionally, the microprocessor 9 modulates the at least one lighting parameter of the lighting units 1, 2 according to predefined formulae taking account of the measured physiological parameters.

The respective physiological parameters are therefore recorded individually and continuously on the patient 7 and used for the biofeedback. The microprocessor 9 is programmed suitably to interpret these physiological parameters and to output a respectively appropriate light pattern via the lighting units 1, 2 to the patient 7, such that a conscious or unconscious biofeedback control loop arises via which the patient 7 may calm his/her vegetative nervous system. Therefore, while the lighting units 1, 2 cause, for example, a pulsating light to influence the patient 7, a change in his/her body reaction in the form of the physiological parameters is measured by the sensors 3, 4, 5 and forwarded to the microprocessor 9. The microprocessor 9 thereupon continuously adapts the light pattern of the lighting units 1, 2 to the changed activation level of the patient 7. The patient 7 perceives these visual stimuli again and regulates his/her nervous activation level consciously or unconsciously by perceiving the effect of the change in the light pattern. The control loop of the biofeedback is closed in this way.

The change in color and/or intensity of the light signals of the lighting units 1, 2 is therefore coupled to the physiological state of the patient 7. The microprocessor 9 adapts the light presentation of the lighting units 1, 2 systematically to the physiological state of the patient 7, such that a biofeedback control loop arises. Corresponding illumination of the lighting units 1, 2 in different colors gives rise to light patterns that influence the vegetative nervous system of the patient 7 by a slow change in the light intensity or the light color (e.g., also pulsation) depending on the physiological state of the patient 7.

The components or assemblies of the user interface are signal-conductively connected to one another in a suitable manner in order to be able to cooperate in accordance with the method. In this case, “signal-conductively” may be not only an electrically conductive connection but also an arbitrary wireless connection. For example, the components or assemblies may also be interconnected via a bus system.

The methods described in detail above and also the arrangement presented are merely exemplary embodiments that may be modified in a variety of ways by the person skilled in the art, without departing from the scope of the invention. Although the invention has been described for use on a computed tomography installation, for example, this does not rule out the advantageous use on other medical-technical installations, such as, for example, other X-ray-based installations (e.g., for creating conventional X-ray recordings or radiological examinations), magnetic resonance imaging apparatuses (MRI), installations for creating images based on radionuclides (e.g., scintigraphy, positron emission tomography (PET), single-photon emission computed tomography (SPECT)), installations for creating images based on ultrasonic waves (e.g., sonography, color Doppler), installations for creating images based on infrared radiation (e.g., diagnostic thermography), installations for creating images based on electrical resistances or impedances (e.g., electrical impedance tomography (EIT)), installations for creating images based on visible light (e.g., endoscopy, optical tomography), and installations for therapeutically irradiating body regions of a patient (e.g., medical linear accelerators).

The use of the indefinite article “a” or “an” does not preclude that the relevant features may also be present repeatedly. The terms “lighting unit”, “microprocessor” and “sensor” do not preclude that the relevant components consist of a plurality of interacting sub-components that, if appropriate, may also be spatially distributed.

Although the invention has been illustrated and described in detail by the exemplary embodiments, the invention is not restricted by the examples disclosed. Other variations may be derived therefrom by the person skilled in the art without departing from the scope of protection of the invention. The described exemplary embodiments, variants, and developments may also be freely combined with one another.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An arrangement for outputting light signals at a medical-technical installation, the arrangement comprising:
a lighting unit having a lighting parameter that is controllable;
a sensor configured to measure a physiological parameter of a patient and to generate a signal depending on the physiological parameter;
a microprocessor configured to evaluate the signal and to control the lighting parameter of the lighting unit depending on the signal; and
a medical-technical installation configured for an examination or treatment of the patient with a different device than the lighting unit and the sensor.

2. The arrangement of claim 1, wherein the microprocessor is configured to:
continuously determine a fluctuating quantitative variable by the evaluation of the signal; and
continuously control the lighting parameter depending on the fluctuating quantitative variable.

3. The arrangement of claim 1, wherein the microprocessor is configured to evaluate the signal in time intervals, for classifying the signal into one from a plurality of categories, and for controlling the lighting parameter depending on the classified category.

4. The arrangement of claim 1, wherein the sensor is configured to measure a rhythmic physiological process, and
wherein the microprocessor is configured to control the lighting parameter depending on a frequency or amplitude of the signal; or
wherein the microprocessor is configured to control the lighting parameter depending on a change in a frequency or an amplitude of the signal; or
wherein the microprocessor is configured to control the lighting parameter depending on a variance of the frequency or the amplitude of the signal.

5. The arrangement of claim 1, wherein the lighting unit is mounted flat on a surface of the medical-technical installation, or
wherein the lighting unit illuminates the surface of the medical-technical installation.

6. The arrangement of claim 1, wherein the lighting parameter is a color temperature, a spectral composition, or a light intensity.

7. The arrangement of claim 1, wherein the sensor is configured to measure a respiration, a heartbeat, a blood pressure, a blood oxygen content, a skin temperature, a skin resistance, a muscle potential, or a brainwave.

8. The arrangement of claim 1, wherein the medical-technical installation is a radiological imaging installation, the radiological imaging installation comprising a computed tomography installation or a C-arm X-ray apparatus, or
wherein the medical-technical installation is an installation for irradiating a patient, the installation for irradiating the patient comprising a medical linear accelerator.

9. A method for outputting light signals at a medical-technical installation, the method comprising:
measuring, by a sensor, a physiological parameter of a patient;
generating, by the sensor, a signal depending on the physiological parameter,
evaluating, by a microprocessor, the signal; and
controlling, by the microprocessor, a lighting parameter of a lighting unit, depending on the signal,
wherein the lighting unit is arranged at or alongside a medical-technical installation configured for an examination or treatment of the patient with a different device than the lighting unit and the sensor.

10. The method of claim 9, wherein the microprocessor is configured to continuously control the lighting parameter depending on a fluctuating quantitative variable determined by the microprocessor by continuous evaluation of the signal.

11. The method of claim 9, wherein the microprocessor is configured to:
evaluate the signal in time intervals;
classify the signal into one from a plurality of categories; and
control the lighting parameter depending on the classified category.

12. The method of claim 9, wherein the sensor is operable to measure a rhythmic physiological process, and
wherein the microprocessor is configured to control the lighting parameter depending on a frequency or an amplitude of the signal, or
wherein the microprocessor is configured to control the lighting parameter depending on a change in the frequency or the amplitude of the signal, or
wherein the microprocessor is configured to control the lighting parameter depending on a variance of the frequency or the amplitude of the signal.

13. The method of claim 9, wherein the lighting parameter is a color temperature, a spectral composition, or a light intensity.

14. The method of claim 9, wherein the sensor measures a respiration, a heartbeat, a blood pressure, a blood oxygen content, a skin temperature, a skin resistance, a muscle potential, or a brainwave.

15. The method of claim 9, wherein the medical-technical installation is a radiological imaging installation, the radiological imaging installation comprising a computed tomography installation or a C-arm X-ray apparatus; or
wherein the medical-technical installation is an installation for irradiating a patient, the medical-technical installation comprising a medical linear accelerator.

16. In a non-transitory computer-readable storage medium having instructions executable by one or more microprocessors to output light signals at a medical-technical installation, the instructions comprising:
measuring, by a sensor, a physiological parameter of a patient;
generating, by the sensor, a signal depending on the physiological parameter,
evaluating, by a microprocessor, the signal; and
controlling, by the microprocessor, a lighting parameter of a lighting unit, depending on the signal,
wherein the lighting unit is arranged at or alongside a medical-technical installation configured for an examination or treatment of the patient with a different device than the lighting unit and the sensor.
